(19) United States
(12) Patent Application Publication
(10) Pub. No.: US 2016/0089391 A1
(43) Pub. Date: Mar. 31, 2016

(54) Molecular Targets for Cardiovascular Disease

(71) Applicant: Temple University-of the Commonwealth System of Higher Education, Philadelphia, PA (US)

(72) Inventor: Hong Wang, Huntingdon Valley, PA (US)

(21) Appl. No.: 14/864,000

(22) Filed: Sep. 24, 2015

Related U.S. Application Data
(60) Provisional application No. 62/057,519, filed on Sep. 30, 2014.

(51) Int. Cl. A61K 31/713; G01N 33/573; A61K 38/06; A61K 45/06; A61K 38/05

(52) U.S. Cl. CPC A61K 31/713; A61K 45/06; A61K 38/05; A61K 38/06; G01N 33/573; G01N 2333/96466; G01N 2800/32

(57) ABSTRACT

The invention relates to methods and compositions for treating and preventing cardiovascular disease, including but not limited to treating and preventing endothelial dysfunction in subjects having hyperhomocysteinemia, hyperglycemia, or a combination of hyperhomocysteinemia and hyperglycemia. As endothelial dysfunction is an indicator for atherosclerosis and cardiovascular disease, this treatment has general impact for early prevention and treatment for all cardiovascular disease. In particular, the present invention relates to methods and compositions for inhibiting calpain activity.

### CROSS-REFERENCE TO RELATED APPLICATIONS

This application is entitled to priority to U.S. Provisional Application No. 62/057,519, filed Sep. 30, 2014, which is incorporated herein by reference in its entirety.

### STATEMENT REGARDING FEDERALLY SPONSORED RESEARCH OR DEVELOPMENT

This invention was made with government support under grant numbers HL67033, HL77288, HL82774, HL110764, HL117654, HL9445, HL108910, HL116917, and SDG16390004 awarded by the National Institutes of Health (NIH). The government has certain rights in the invention.

### BACKGROUND OF THE INVENTION

Cardiovascular disease (CVD) is one of the most prevalent complications and a major cause of premature mortality in patients with diabetes. Numerous factors have been suggested to be related to CVD in diabetes, such as hyperinsulinemia, hyperlipidemia, hyperglycemia, obesity, and smoking.

Accumulative evidence indicate that hyperhomocysteinemia (HHcy), referring to elevated concentrations of a sulfur-containing amino acid plasma homocysteine (Hcy) produced from methionine metabolism, is also linked to CVD in diabetes.

HHcy has been established as an independent and significant risk factor for CVD (McCully, 1969, Am J Pathol 56:111-128). Recent studies have shown a high prevalence of HHcy in patients with diabetes, and the plasma concentration of Hcy is positively correlated to macrovascular diseases (Smulders et al., 1999, Diabetes care 22:125-132), cardiovascular morbidity and mortality (Hofmann et al., 1998, Diabetes care 21:841-848) in diabetes. Whether HHcy aggravates CVD in diabetes and how the underlying mechanisms work, however, remain incompletely understood.

Endothelial dysfunction (ED) is an early event in the development of CVD, occurring before morphological changes in the vasculature can be detected. ED is defined by reduced endothelium-dependent vascular relaxation to acetylcholine (ACh) (Furchgott et al., 1989, Official Publication of the Federation of American Societies for Experimental Biology 3:2007-2018). In humans, ED has been observed in patients with high plasma Hcy levels (usually >100 μmol/L) (Celermajer et al., 1993, J Am Coll Cardiol 22:854-858). It has been reported that HHcy impairs endothelium-dependent relaxation in mouse aortas, cremaster microvasculature, and small mesenteric arteries (Jiang et al., 2005, Arterioscl Throm Vas 25:2515-2521; Cheng et al., 2011, Blood 118:1998-2006). Also reported are HHcy-induced ED, protein kinase C (PKC)-induced phosphorylation of endothelial nitric oxide synthase (eNOS) at threonine 495 (PKC-eNOSp-Thr495), and eNOS inactivation in mouse thoracic aortas (Jiang et al., 2005, Arterioscl Throm Vas 25:2515-2521).

PKC is an important signaling molecule associated with ED in diabetes, and a substrate of calpain, a family of calcium-dependent cysteine-proteases which tightly regulate their respective substrates through limited proteolytic cleavage (Inoue et al., 1977, J Biol Chem 252:7610-7616, Takai et al., 1977, Biochem Bioph Res Co 77:542-550). In the calpain family, μ- and m-calpain are well-characterized and abundantly expressed in ECs (Goll et al., 2003, Physiol Rev 83:731-801). A large number of proteins are cleaved by the calpains in in vitro and in vivo (Goll et al., 2003, Physiol Rev 83:731-801). Calpain cleaves PKC, leading to PKC activation constitutively and induces a variety of signal transduction process (Inoue et al., 1977, J Biol Chem 252:7610-7616, Takai et al., 1977, Biochem Bioph Res Co 77:542-550).

Activation of calpain can promote platelet aggregation, neurovascular dysfunction, and cardiomyocyte apoptosis in diabetes (Randriamboavonjy et al., 2008, Circulation 117:52-60; Nangle et al., 2006, Eur J Pharmacol 538:148-153). Recent studies have shown that activation of calpain regulates acute and chronic hyperglycemia (HG)-induced leukocytes-endothelium integration in rat mesenteric arteries (Stalker et al., 2005, Diabetes 54:1132-1140; Stalker et al, 2003, Official Publication of the Federation of American Societies for Experimental Biology 17:1511-1513). Increased calpain activity was also found in primary hepatocytes of Cbs^{-/+} mice and Hcy-treated cultured rat heart microvascular ECs (Hamelet et al., 2009, Mol Genet Metab 92:114-120; Moshal et al., 2006, Am J Physiol Heart Circ Physiol 291:H2825-2835).

There is a need in the art for a molecular target in HHcy- and HHcy/HG-related ED. The present invention addresses this unmet need.

### SUMMARY OF THE INVENTION

The present invention relates to methods and compositions for treating and preventing cardiovascular disease, including but not limited to treating and preventing endothelial dysfunction in subjects having hyperhomocysteinemia, hyperglycemia, or a combination of hyperhomocysteinemia and hyperglycemia.

In one aspect, the invention relates to a method for treating endothelial dysfunction in a subject having hyperhomocysteinemia, hyperglycemia, or a combination of hyperhomocysteinemia and hyperglycemia, comprising administering to the subject a therapeutically effective amount of a composition comprising at least one inhibitor of calpain. In one embodiment, the inhibitor is selected from the group consisting of: a small interfering RNA (siRNA), a microRNA, an antisense nucleic acid, a ribozyme, an expression vector encoding a transdominant negative mutant, an antibody, a peptide, a chemical compound, and a small molecule.

In one embodiment, the peptide is at least one selected from the group comprising MDL28170, calpeptin, and ALLM. In one embodiment, the siRNA is μ-calpsiRNA. In one embodiment, the composition further comprises a pharmaceutically acceptable excipient. In one embodiment, the composition is administered in combination with another therapeutic agent. In one embodiment, the therapeutic agent is an antioxidant. In one embodiment, the antioxidant is at least one selected from the group comprising PEG-SOD, Tempol, and Apocynin. In one embodiment, the therapeutic agent is a PKC suppressor. In one embodiment, the PKC suppressor is at least one selected from the group comprising GFX and dominant negative PKCβ2 adenovirus.

In another aspect, the invention relates to a method for detecting the onset of cardiovascular disease in a subject having hyperhomocysteinemia or a combination of hyperhomocysteinemia and hyperglycemia. The method comprises the steps of obtaining in a biological sample the quantity of calpain N-terminal catalytic subunits, obtaining in the same biological sample the quantity of total calpain protein levels, and transforming the quantities obtained into a therapeutically relevant ratio of calpain N-terminal catalytic subunits to total calpain protein levels.

In one embodiment, the quantity of catalytic subunits obtained is the quantity of N-terminal catalytic subunits of μ-calpain and the quantity of total calpain protein level obtained is the quantity of total μ-calpain protein level. In one embodiment, the ratio is less than 40%. In another embodiment, the ratio is less than 20%. In another embodiment, the ratio is less than 10%. In another embodiment, the ratio is less than 5%. In another aspect, the invention relates to a kit for diagnosing or monitoring the onset of cardiovascular disease in a subject, wherein the quantity of calpain N-terminal catalytic subunits is obtained from a biological sample from said subject, the quantity of total calpain protein levels is obtained from the same biological sample from said subject, and the quantities obtained are transformed into a therapeutically relevant ratio of calpain N-terminal catalytic subunits to total calpain protein levels. In one embodiment, the quantity of catalytic subunits obtained is the quantity of N-terminal catalytic subunits of μ-calpain and the quantity of total calpain protein level obtained is the quantity of total μ-calpain protein level.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A through FIG. 1C depict the results of experiments demonstrating that mice develop HHcy and/or HG by feeding a high methionine diet and/or injection of streptozotocin. (FIG. 1A). Total plasma homocysteine (tHcy) levels. (FIG. 1B). Blood glucose levels. (FIG. 1C). Body weight. At the age of 8-week, Cbs^{+/+} and Cbs^{-/+} mice were fed with a high methionine diet (HM, methionine: 2% w/w) for 8 weeks, and/or injected with streptozotocin (STZ, i.p., 40 mg/kg) for 5 consecutive days starting from the first day of experiment. Mice fed with a control diet (CT, methionine: 0.37% w/w) and injected with vehicle (50 mmol/L citrate buffer) served as controls. Total plasma Hcy (t-Hcy) level was measured by mass spectrometer (Baylor Research Institute, 3812 Elm Street, Dallas, Tex. 75226). Blood samples for blood glucose were collected via a tail clip protocol and blood glucose levels were determined with an automatic glucometer (HemoCue AB). n=5-10, *p<0.05 vs vehicle-treated corresponding mice on CT diet; †p<0.05 vs vehicle-treated corresponding mice on HM diet; ‡p<0.05 vs STZ-treated corresponding mice on CT diet. Cbs, cystathionine β-synthase; HG, hyperglycemia; HHcy, hyperhomocysteinemia; i.p. intraperitoneal.

FIG. 2A through FIG. 2C depict the results of experiments demonstrating that HHcy aggravates HG-induced endothelial dysfunction (ED), endothelium-dependent vascular relaxation to Ach, in mouse thoracic aorta (FIG. 2A) via nitric oxide as it was diminished in the presence of NOS inhibitor N^{G}-nitro-L-arginine methyl ester (L-NAME, 100 μmol/L, 30 min) (FIG. 2B), and as endothelium-independent vascular relaxation to SNP was not changed (FIG. 2C).

FIG. 3A and FIG. 3B depict the results of experiments demonstrating that activation of calpain regulates HHcy/HG-induced ED in mouse aorta. Images (Left Panel) and quantification (Right Panel) showing HHcy and HG activates calpain in mouse aortic endothelial cells (MAECs, P₀) isolated from CBS^{-/+} mice (FIG. 3A). Calpain activity was measured in intact MAECs by the fluorescent substrate t-BOC-Leu-Met-CMAC (T-BOC, 10 μmol/L, 30 min). In FIG. 3B, calpain inhibitors rescued endothelium-dependent vascular relaxation to ACh in the aorta of HHcy CBS^{-/+} and HHcy/HG CBS^{-/+} mice in the presence of calpain inhibitors in vivo (Left Panel) and ex vivo (Right Panel). Left Panel: Calpain inhibitor MDL28170 (MDL, Sigma #M6690) was administrated in vivo by i.p. injection (2 mg/kg/day, 2 weeks). Right Panel: The arterial rings were incubated with calpain inhibitors: MDL, calpeptin and N-Acetyl-Leu-Leu-Met-CHO (ALLM, 20 μmol/L for all, Enzo life Sciences, PI-100-0005) for 1 hr. Aortic arterial rings were pre-contracted with phenylephrine (1 μmol/L) and examined for relaxation response to accumulative concentrations of ACh. n=3-10, *p<0.05 vs vehicle-treated Cbs^{-/+} mice on CT diet; †p<0.05 vs vehicle-treated Cbs^{-/+} mice on HM diet; ‡p<0.05 vs STZ-treated Cbs^{-/+} mice on CT diet; #p<0.05 vs STZ-treated Cbs^{-/+} mice on HM diet. Cbs, cystathionine β-synthase; ED, endothelial dysfunction; HG, hyperglycemia; HHcy, hyperhomocysteinemia.

FIG. 4A through FIG. 4F depict the results of experiments demonstrating that μ-calpain activation regulates HHcy/HG-induced ED in mouse aorta. (FIG. 4A). Structure and activation of μ/m-calpain large subunit. Inverse μ- and m-calpain activity was assessed by immunoblot analysis using a primary antibody that recognizes the μ- or m-calpain large subunit (catalytic subunit) NT domain, which is autolyzed in active calpains. A primary antibody that recognizes the μ- or m-calpain IV domains represent total calpain levels. Protein extract (40 μg) from mouse aorta was used for Western blot for detecting p/m-calpain activity and protein expression. (FIG. 4B). Inverse μ-calpain activity. (FIG. 4C). Inverse m-calpain activity. (FIG. 4D). Total μ-calpain protein levels. (FIG. 4E). Total m-calpain protein levels. (FIG. 4F). Endothelium-dependent vascular relaxation to acetylcholine (ACh) in Cbs^{+/+} mouse aorta in the presence of μ-calsiRNA. The thoracic aorta from control Cbs^{+/+} mice was incubated in endothelial grow medium and treated with or without control siRNA (CTsiRNA) or μ-calpain siRNA μ-calpsiRNA) for 22 hrs followed with additions of DL-Hcy (500 μmol/L) and D-Glu (25 mmol/L) in ECGM for 48 hrs. Aorta treated with DL/Hcy/D-Glu in the absence of siRNA served as control. Aortic arterial rings were pre-contracted with phenylephrine (1 μmol/L) and examined for relaxation response to cumulative additions of ACh. n=3-5, #p<0.05 vs aorta treated with DL-Hcy/D-Glu. Cbs, cystathionine β-synthase; CTsiRNA, control siRNA; ED, endothelial dysfunction; HG, hyperglycemia; HHcy, hyperhomocysteinemia; m-calp, m-calpain; μ-calp, μ-calpain; μ-calpsiRNA, μ-calpain siRNA.

FIG. 5A through FIG. 5F depict the results of experiments demonstrating that oxidative stress mediates HHcy/HG-induced ED and calpain activation. (FIG. 5A). In situ .O₂⁻ production in aorta. (FIG. 5B). Nitrotyrosine (3-NT) levels. Aortic paraffin-cross sections (7 μm) from Cbs^{-/+} mice were stained with 3-NT antibody and 3,3′-diaminobenzidine (DAB, brown). (FIG. 5C). Urinary 8-isoprostane levels. At the age of 8-week, Cbs^{+/+} and Cbs^{-/+} mice were fed with a high methionine diet (HM, methionine: 2% w/w) for 8 weeks, and/or injected with streptozotocin (STZ, i.p., 40 mg/kg) for 5 consecutive days starting from the first day of experiment. Mice fed with a control diet (CT, methionine: 0.37% w/w) and injected with vehicle (50 mM citrate buffer) served as controls. Aortic cryo-cross sections (30 μm) from Cbs^{-/+} mice were stained with the superoxide marker dihydroethidium (DHE, 3 μmol/L, 30 mins.). Urine was collected from 24 hrs metabolic cage. (FIG. 5D). Endothelium-dependent vascular relaxation in HHcy/HG CBS^{-/+} mouse aorta in the presence of antioxidants. Arterial rings were pre-contracted with phenylephrine (1 μmol/L) and examined for relaxation to cumulative additions of ACh in the aorta of STZ-treated Cbs^{-/+} mice on HM diet. The arterial rings were preincubated with antioxidants polyethylene glycol superoxide dismutase (PEG-SOD, 150 U/mL, Sigma #S9549), Tempol (1 mmol/L, Sigma #176141), or apocynin (10 μmol/L, Sigma #W508454) for 1 hr. n=3-5. *p<0.05 vs vehicle-treated Cbs^{-/+} mice on CT diet; †p<0.05 vs vehicle-treated Cbs^{-/+} mice on HM diet; ‡p<0.05 vs STZ-treated Cbs^{-/+} mice on CT diet; #p<0.05 vs STZ-treated Cbs^{-/+} mice on HM diet. (FIG. 5E). Calpain activity in HAECs. Calpain activity was measured by the fluorescent substrate t-BOC-Leu-Met-CMAC (T-BOC, 10 μmol/L, 30 min) Human aortic endothelial cells (HAECs, passage 8-9) were cultured in EC medium which contains 5 mmol/L D-glucose (D-Glu), treated with DL-homocysteine (DL-Hcy, 500 μmol/L) and/or D-Glu (25 mmol/L) with or without calpain inhibitor PEG-SOD (150 U/mL) or MDL28170 (MDL, 20 μmol/L) for 48 hrs, or CTsiRNA and μ-calpsiRNA for 72 hrs. HAECs were cultured in EC medium with or without control siRNA (CT siRNA, Santa Cruz #sc-37007) or μ-calpain (μ-calpsiRNA, Santa Cruz #sc-29885) for 48 hrs. Transfection effect of μ-calpsiRNA in HAECs was detected by Western blot after transfected with the siRNA for indicated time. (FIG. 5F). Transfection effect of μ-calpain siRNA in HAECs. n=3, *p<0.05 vs control HAECs; †p<0.05 vs DL-Hcy-treated HAECs; #p<0.05 vs D-Glu-treated HAECs; ‡p<0.05 vs DL-Hcy/D-Glu-treated HAECs. Cbs, cystathionine β-synthase; CTsiRNA, control siRNA; ED, endothelial dysfunction; HG, hyperglycemia; HHcy, hyperhomocysteinemia; μ-calpsiRNA, μ-calpain siRNA.

FIG. 6A through FIG. 6C depict the results of experiments demonstrating that μ-calpain mediates HHcy/HG-induced NO reduction and eNOS inactivation in HAECs. (FIG. 6A). Images of NO. Nitric oxide (NO) production in HAECs was measured by staining intact HAECs with NO marker DAF-FM. N^{G}-nitro-L-arginine methyl ester (L-NAME, 100 μmol/L, Sigma #N5751) was added in the last hour as a negative control. (FIG. 6B). Quantification of NO. (FIG. 6C). eNOS activity. Human aortic endothelial cells (HAECs) were cultured in EC medium which contains 5 mmol/L D-Glucose (D-Glu), and treated with DL-homocysteine (DL-Hcy, 500 μmol/L) and/or (D-Glu, 25 mmol) without or with MDL28170 (MDL) for 48 hrs, CTsiRNA or μ-calpsiRNA for 72 hrs. L-NAME (100 μmol/L) was added in the last hour. NO was measured by staining intact HAECs with NO marker (DAF-FM, 10 μmol/L, Sigma #D2321, 30 mins.). Endothelial nitric oxide synthase (eNOS) activity was assessed by measuring of ³H-L-arginine conversion to ³H-citrulline in intact HAECs. n=3, *p<0.05 vs control; †p<0.05 vs DL-Hcy-treated HAECs; ‡p<0.05 vs D-Glu-treated HAECs; #p<0.05 vs DL-Hcy/D-Glu-treated HAECs. DAF-FM, 4-Amino-5-methylamino-2′,7′-difluorescein; CTsiRNA, control siRNA; HG, hyperglycemia; HHcy, hyperhomocysteinemia; μ-calpsiRNA, μ-calpain siRNA.

FIG. 7A through FIG. 7D depict the results of experiments demonstrating that activation of μ-calpain and PKCβ₂ mediates HHcy/HG-induced phosphorylation of eNOSThr497/495. (FIG. 7A). eNOS protein levels in mouse aorta of Cbs^{-/+} mice (Left Panel) and HAECs (Right Panel). (FIG. 7B). eNOS-pThr497/495 protein levels in mouse aorta of Cbs^{-/+} mice (Left Panel) and in HAECs (Right Panel). At the age of 8-week, Cbs^{-/+} mice were fed with a high methionine diet (HM, methionine: 2% w/w) for 8 weeks, and/or injected with streptozotocin (STZ, i.p., 40 mg/kg) for 5 consecutive days starting from the first day of experiment. Mice fed with a control diet (CT, methionine: 0.37% w/w) and injected with vehicle (50 mmol/L citrate buffer) served as controls. Human aortic endothelial cells (HAECs, passage 8-9) were cultured in EC medium which contains 5 mmol/L D-glucose (D-Glu), treated with DL-homocysteine (DL-Hcy, 500 μmol/L) and/or D-Glu (25 mmol/L). (FIG. 7C). eNOS-pThr495 protein levels in HAECs. HAECs (passage 8-9) were cultured in EC medium which contains 5 mmol/L D-glucose (D-Glu), treated with DL-homocysteine (DL-Hcy, 500 μmol/L) and D-Glu (25 mmol/L for 48 hrs with or without non-specific PKC inhibitor GF109203X (GFX) (2 μmol/L, 30 min.); control adenoviruse (Ad-CT, 200 MOI, 72 hrs), dominant negative PKCβ₂ adenovirus (Adv-dnPKCβ₂, 200 MOI, 72 hrs); μ-calpain siRNA (μ-calpsiRNA, Santa Cruz #sc-29885, 72 hrs), or MDL (20 mmol/L, 48 hrs, Sigma #M6690). (FIG. 7D). Transfection effect of Adv-dnPKCβ₂in HAECs. Cultured HAECs (passage 8, 80% confluence) were treated with 12.5, 25, 50, 100 and 200 MOI Adv-dnPKCβ₂ for 48 hrs. n=3, *p<0.05 vs control; †p<0.05 vs DL-Hcy-treated HAECs; ‡p<0.05 vs D-Glu-treated HAECs; #p<0.05 vs DL-Hcy/D-Glu-treated HAECs. Cbs, cystathionine β-synthase; eNOS, endothelial nitric oxide synthase; eNOS-pThr495/497, phosphorylation of eNOS at threonine 497 (mouse)/495 (human).

FIG. 8 depicts the mechanisms of HHcy/HG-induced ED. Adv-dnPKCβ₂ dominant negative PKCβ₂ adenovirus; ALLM, N-Acetyl-Leu-Leu-Met-CHO; ED, endothelial dysfunction; eNOS, endothelial nitric oxide synthase; eNOS-pThr495/497, GFX, GF109203X; HHcy, hyperhomocysteinemia; HG, hyperglycemia; PEG-SOD, polyethylene glycol superoxide dismutase; phosphorylation of eNOS at threonine 495/497; NO, nitric oxide; O₂⁻, superoxide.

FIG. 9A through FIG. 9C depict the results of experiments demonstrating that HHcy aggravates HG-induced ED in mouse thoracic aorta mainly via nitric oxide. At the age of 8-week, Cbs^{+/+} and Cbs^{-/+} mice were fed with a high methionine diet (HM, methionine: 2% w/w) for 8 weeks, and/or injected with streptozotocin (STZ, i.p., 40 mg/kg) for 5 consecutive days starting from the first day of experiment. Mice fed with a control diet (CT, methionine: 0.37% w/w) and injected with vehicle (50 mM citrate buffer) served as controls. (FIG. 9A). Endothelium-dependent vascular relaxation to ACh. (FIG. 9B) Endothelium-dependent vascular relaxation to ACh in the presence of NOS inhibitor N^{G}-nitro-L-arginine methyl ester (L-NAME, 100 μmol/L, 30 min). (FIG. 9C). Endothelium-independent vascular relaxation to SNP. Aortic arterial rings were pre-contracted with phenylephrine (1 μmol/L) and examined for relaxation response to cumulative additions of acetylcholine (ACh) or sodium nitroprusside (SNP). Endothelium-dependent relaxation to ACh and SNP was represented as the area under the curve (AUC). n=5-10, *p<0.05 vs vehicle-treated corresponding mice on CT diet; †p<0.05 vs vehicle-treated corresponding mice on HM diet; ‡p<0.05 vs STZ-treated corresponding mice on CT diet. Cbs, cystathionine β-synthase; ED, endothelial dysfunction; HG, hyperglycemia; HHcy, hyperhomocysteinemia; i.p. intraperitoneal.

FIG. 10 depicts the results of experiments demonstrating the identification of mouse aortic endothelial cells (MAEC). Cultured MAECs isolated from control Cbs^{-/+} mice (p0) were identified by endothelial makers vWF (red, Von Willebrand factor, vWF, Santa Cruz SC-2780, 1:200)) and CD31 (green, CD31 (BD Biosciences; Bd553370, 1:100). Endothelial cell were stained with DAPI (blue, 4′,6-Diamidino-2-Phenylindole).

FIG. 11 depicts the results of experiments demonstrating that activation of calpain regulates HHcy/HG-induced ED in mouse aorta. Endothelium-dependent vascular relaxation to ACh in the presence of calpain inhibitors in vivo (Left Panel) and ex vivo (Right Panel). At the age of 8-week, Cbs^{-/+} mice were fed with a high methionine diet (HM, methionine: 2% w/w) for 8 weeks, and/or injected with streptozotocin (STZ, i.p., 40 mg/kg) for 5 consecutive days starting from the first day of experiment. Mice fed with a control diet (CT, methionine: 0.37% w/w) and injected with vehicle (50 mmol/L citrate buffer) served as controls. Left Panel: Calpain inhibitor MDL28170 (MDL) was administrated in vivo by i.p. injection (2 mg/kg/day, 2 weeks). Right Panel: The arterial rings were incubated with calpain inhibitors: MDL, calpeptin and ALLM (N-Acetyl-Leu-Leu-Met-CHO, 20 μmol/L for all) for 1 hr. Aortic arterial rings were pre-contracted with phenylephrine (1 μmol/L) and examined for relaxation response to accumulative concentrations of ACh. Endothelium-dependent relaxation to ACh was represented as the area under the curve (AUC). n=3-10, *p<0.05 vs vehicle-treated Cbs^{-/+} mice on CT diet; †p<0.05 vs vehicle-treated Cbs^{-/+} mice on HM diet; ‡p<0.05 vs STZ-treated Cbs^{-/+} mice on CT diet; #p<0.05 vs STZ-treated Cbs^{-/+} mice on HM diet. Cbs, cystathionine β-synthase; ED, endothelial dysfunction; HG, hyperglycemia; HHcy, hyperhomocysteinemia.

FIG. 12 depicts the results of experiments demonstrating that μ-calpain siRNA rescues HHcy/HG-induced ED. Endothelium-dependent vascular relaxation to acetylcholine (ACh) in mouse aorta treated with DL-Hcy and/or D-Glu in vitro. The thoracic aorta from control Cbs^{|/|} mice was incubated in endothelial grow medium treated with or without control siRNA (CTsiRNA) or μ-calpain siRNA (μ-calpsiRNA) for 16 hours then DL-homocysteine (DL-Hcy, 500 μmol/L) plus D-glucose (D-Glu, 25 mmol/L) for were added into the medium for 48 hours. Aorta treated with DL/Hcy/D-Glu in the absence of siRNA served as control. Aortic arterial rings were pre-contracted with phenylephrine (1 μmol/L) and examined for relaxation response to cumulative additions of ACh. Endothelium-dependent relaxation to ACh was represented as the area under the curve (AUC). n=3-5, *p<0.05 vs vehicle-treated corresponding mice on CT diet; †p<0.05 vs vehicle-treated corresponding mice on HM diet; ‡p<0.05 vs STZ-treated corresponding mice on CT diet. #p<0.05 vs STZ-treated corresponding mice on HM diet. Cbs, cystathionine β-synthase; ED, endothelial dysfunction; HG, hyperglycemia; HHcy, hyperhomocysteinemia; μ-calp, μ-calpain.

FIG. 13 depicts the results of experiments demonstrating that oxidative stress regulates HHcy/HG-induced ED. Endothelium-dependent vascular relaxation to accumulative concentrations of acetylcholine in aorta of Cbs^{-/|} mice. At the age of 8-week, Cbs^{-/+} mice were fed with a high methionine diet (HM, methionine: 2% w/w) for 8 weeks and with injection of streptozotocin (STZ, i.p., 40 mg/kg) for 5 consecutive days starting from the first day of experiment. Arterial rings were pre-contracted with phenylephrine (1 μmol/L) and examined for relaxation to cumulative additions of ACh in the mouse aorta. The arterial rings were preincubated with antioxidants polyethylene glycol superoxide dismutase (PEG-SOD, 150 U/mL), Tempol (1 mmol/L), or apocynin (10 μmol/L) for 1 hr. n=3-5. #p<0.05 vs STZ-treated Cbs^{-/+} mice on HM diet. Cbs, cystathionine β-synthase; ED, endothelial dysfunction; HG, hyperglycemia; HHcy, hyperhomocysteinemia.

### DETAILED DESCRIPTION

The present invention is partly based upon the discovery that calpain activity contributes to endothelial dysfunction and cardiovascular disease in subjects having hyperhomocysteinemia, hyperglycemia, or a combination of hyperhomocysteinemia and hyperglycemia. The results presented herein demonstrate a causal link between calpain activation and reduced endothelium-dependent vascular relaxation to acetylcholine.

In one embodiment, the present invention is directed to methods and compositions for treatment, inhibition, prevention, or reduction of cardiovascular disease. In another embodiment, the present invention is directed to methods and compositions for treatment, inhibition, prevention, or reduction of endothelial dysfunction. In one embodiment, the invention provides compositions and methods for inhibiting one or more of the level, production, and activity of calpain.

In one embodiment, the present invention comprises a method for decreasing one or more of the level, production, and activity of calpain comprising administering to a subject an effective amount of a composition comprising an inhibitor of calpain. In an embodiment of the present invention, the composition decreases the transcription of calpain or translation of calpain mRNA. In another embodiment of the present invention, the composition inhibits the activity of calpain.

Accordingly, the invention provides inhibitors of calpain activity. In one embodiment, the invention provides inhibitors of calpain upstream activity. In one embodiment, the invention provides inhibitors of calpain downstream activity. In one embodiment, the inhibitor of calpain activity includes but is not limited to a small molecule, a chemical compound, a protein, a peptide, a peptidomemetic, a nucleic acid, and the like.

Another aspect of the present invention comprises a pharmaceutical composition comprising an inhibitor of calpain. In one embodiment, the composition of the invention can be used in combination with another therapeutic agent. In one embodiment, the therapeutic agent is an antioxidant. In another embodiment, the therapeutic agent is a PKC suppressor.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

By the term “specifically binds,” as used herein, is meant a molecule, such as an antibody or a small molecule, which recognizes and binds to another molecule or feature, but does not substantially recognize or bind other molecules or features in a sample.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “calpain inhibitor” refers to an agent that inhibits or reduces the activity of calpain or the expression of a calpain gene. A “calpain inhibitor” can inhibit or reduce calpain activity and inhibits or reduce expression of a calpain gene. Calpains that can inhibited by the methods and compositions described herein, include, but are not limited to a full-length calpain, a calpain homolog, a calpain variant, a calpain analog, a mutant calpain, a calpain fusion protein, or a calpain peptide mimetic. Exemplary calpain inhibitors include peptides, peptidomimetics, small molecules, compounds, agents, dominant negative mutants of calpain activity, ligand mimetics, antibodies (e.g., monoclonal, polyclonal or single chain Fv; intact or binding fragments thereof), or nucleic acids (e.g., RNA, DNA, antisense oligonucleotides, double stranded RNA oligonucleotides (RNAi) or DNA oligonucleotides (vectors) containing nucleotide sequences encoding for the transcription of shRNA molecules) or derivatives and analogs thereof. Further exemplary calpain inhibitors are disclosed herein.

Administration of the calpain inhibitors described herein can be performed according to any method known to one skilled in the art. For example, when administered to a subject, the administration step can comprise directly contacting airway epithelial cells with a calpain inhibitor. The administration step can also comprise transport or diffusion of the calpain inhibitor across one of more biological membranes in a cell. Many suitable methods for the delivery of a calpain inhibitor to a site in a subject are known in the art. These methods include, but are not limited to, administration of a calpain inhibitor to a subject, wherein the administration step comprises a step of delivering the calpain inhibitor into the airway of the subject by inhalation with the use of a nebulizer. One skilled in the art will also recognize that exist many different methods suitable for use with the methods described herein for delivery of a therapeutic compound, for example a calpain inhibitor, to a selected epithelial layer in a subject. For example, creams, gels, ointments, emulsions, solutions, elixirs, lotions, suspensions, tinctures, pastes, foams, aerosols, irrigations, sprays, nebulized formulation, suppositories, bandages, dermal patches or any other formulations suitable for topical administration or any other method of delivery, including, but not limited to those described herein, can be used can be used to deliver a calpain inhibitor in conjunction with the methods described herein.

As used herein, an “antioxidant” is a substance that, when present in a mixture containing an oxidizable substrate biological molecule, significantly delays or prevents oxidation of the substrate biological molecule. Antioxidants can act by scavenging biologically important reactive free radicals or other reactive oxygen species (.O2⁻, H₂O₂, .OH, HOCl, ferryl, peroxyl, peroxynitrite, and alkoxyl), or by preventing their formation, or by catalytically converting the free radical or other reactive oxygen species to a less reactive species.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell. “Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, a “recombinant cell” is a host cell that comprises a recombinant polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration. “Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

“Sample” or “biological sample” as used herein means a biological material from a subject, including but is not limited to organ, tissue, exosome, blood, plasma, saliva, urine and other body fluid. A sample can be any source of material obtained from a subject.

By the term “specifically binds,” as used herein, is meant a molecule, such as an antibody, which recognizes and binds to another molecule or feature, but does not substantially recognize or bind other molecules or features in a sample.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

The term “treat” or “treating”, as used herein, means reducing the frequency with which symptoms are experienced by a subject or administering an agent or compound to reduce the frequency and/or severity with which symptoms are experienced. As used herein, “alleviate” is used interchangeably with the term “treat.” Treating a disease, disorder or condition may or may not include complete eradication or elimination of the symptom.

As used herein, the term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In one embodiment, the invention provides an inhibitor of calpain. In various embodiments, the present invention includes compositions for inhibiting the level or activity of calpain in a subject, a cell, a tissue, or an organ in need thereof. In various embodiments, the compositions of the invention inhibit the amount of polypeptide of calpain, the amount of mRNA of calpain, the amount of activity of calpain, or a combination thereof In various embodiments, the compositions of the invention inhibit downstream targets of calpain.

The compositions of the invention include compositions for treating or preventing endothelial dysfunction and cardiovascular diseases. In one embodiment of the invention, an inhibitor of calpain is useful for treating endothelial dysfunction. In one embodiment of the invention, an inhibitor of calpain is useful for treating cardiovascular disease.

### Calpain Inhibitors

In various embodiments, the present invention includes compositions and methods of treating endothelial dysfunction and cardiovascular disease in a subject. In various embodiments, the composition for treating endothelial dysfunction and cardiovascular disease comprises an inhibitor of calpain. In one embodiment, the inhibitor of the invention decreases the amount of calpain polypeptide, the amount of calpain mRNA, the amount of calpain activity, or a combination thereof.

It will be understood by one skilled in the art, based upon the disclosure provided herein, that a decrease in the level of calpain encompasses the decrease in the expression, including transcription, translation, or both. The skilled artisan will also appreciate, once armed with the teachings of the present invention, that a decrease in the level of calpain includes a decrease in the activity of calpain. Thus, decrease in the level or activity of calpain includes, but is not limited to, decreasing the amount of polypeptide of calpain, and decreasing transcription, translation, or both, of a nucleic acid encoding calpain; and it also includes decreasing any activity of calpain as well.

In one embodiment, the invention provides a concept for inhibiting calpain as a therapy for cardiovascular disease. In one embodiment, the composition of the invention comprises an inhibitor of calpain. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of calpain in a cell is by reducing or inhibiting expression of the nucleic acid encoding calpain. Thus, the protein level of calpain in a cell can also be decreased using a molecule or compound that inhibits or reduces gene expression such as, for example, siRNA, an antisense molecule or a ribozyme. However, the invention should not be limited to these examples.

In one embodiment, siRNA is used to decrease the level of calpain. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of calpain at the protein level using RNAi technology. None limiting examples of RNAi molecules useful to decrease levels of calpain include those disclosed in Ran et al., 2011 J Molecular Cell Biology 3: 283-292.

In other related aspects, the invention includes an isolated nucleic acid encoding an inhibitor, wherein an inhibitor such as an siRNA or antisense molecule, inhibits calpain, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein. In another aspect of the invention, calpain or a regulator thereof, can be inhibited by way of inactivating and/or sequestering one or more of calpain, or a regulator thereof. As such, inhibiting the effects of calpain can be accomplished by using a transdominant negative mutant.

In another aspect, the invention includes a vector comprising an siRNA or antisense polynucleotide. Preferably, the siRNA or antisense polynucleotide is capable of inhibiting the expression of calpain. The incorporation of a desired polynucleotide into a vector and the choice of vectors is well-known in the art as described in, for example, Sambrook et al., supra.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit calpain. The antisense expressing vector is used to transfect a mammalian cell or the mammal itself, thereby causing reduced endogenous expression of calpain.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Compositions and methods for the synthesis and expression of antisense nucleic acids are as described elsewhere herein.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit calpain. Ribozymes useful for inhibiting the expression of a target molecule may be designed by incorporating target sequences into the basic ribozyme structure which are complementary, for example, to the mRNA sequence of calpain of the present invention. Ribozymes targeting calpain may be synthesized using commercially available reagents (Applied Biosystems, Inc., Foster City, Calif.) or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In another aspect of the invention, calpain can be inhibited by way of inactivating and/or sequestering calpain. As such, inhibiting the effects of calpain can be accomplished by using a transdominant negative mutant. Alternatively an antibody specific for calpain (e.g., an antagonist to calpain) may be used. In one embodiment, the antagonist is a protein and/or compound having the desirable property of interacting with a binding partner of calpain and thereby competing with the corresponding protein. In another embodiment, the antagonist is a protein and/or compound having the desirable property of interacting with calpain and thereby sequestering calpain.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that the present invention includes the use of a single antibody recognizing a single antigenic epitope but that the invention is not limited to the use of a single antibody. Instead, the invention encompasses the use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)2 fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

### Treatment Methods

In one embodiment, the present invention provides methods for treatment, inhibition, prevention, or reduction of endothelial dysfunction using an inhibitor of calpain. In another embodiment, the present invention provides methods for treatment, inhibition, prevention, or reduction of cardiovascular disease using an inhibitor of calpain.

The present invention provides methods of treating or preventing endothelial dysfunction. Endothelial dysfunction is generally a state (e.g., a systemic state) in a subject which is characterized by an imbalance between vasodilating and vasoconstricting substances and/or which is characterized by a shift in the action of endothelium toward reduced vasodilation and/or a proinflammatory state and/or and prothrombic properties. Methods for detecting endothelial dysfunction will be apparent to the skilled person and/or described herein.

The general approach to decreasing calpain activity according to the present invention is to provide a cell with a calpain inhibitor. In one embodiment, the calpain inhibitor may be delivered directly, a preferred embodiment involves providing a nucleic acid encoding a calpain inhibitor, i.e., a calpain siRNA, to the cell. Following this provision, the calpain siRNA silences the host cell's transcriptional and translational machinery for calpain, as well as any that may be provided by the expression construct.

In order to effect inhibition of constructs encoding calpain and other calpain-like genes, the inhibition construct must be delivered into a cell. One mechanism for delivery is via viral infection, where the inhibition construct is encapsidated in a viral particle which will deliver either a replicating or non-replicating nucleic acid.

In another embodiment of the invention, the inhibition construct may simply consist of naked recombinant DNA or plasmids. Transfer of the construct may be performed by any of the methods mentioned above which physically or chemically permeabilize the cell membrane. This is particularly applicable for transfer in vitro, but it may be applied to in vivo use as well. Another embodiment of the invention for transferring a naked DNA expression construct into cells may involve particle bombardment. This method depends on the ability to accelerate DNA coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In a further embodiment of the invention, the inhibition construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Other inhibition constructs which can be employed to deliver a nucleic acid encoding a calpain silencer into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

In certain embodiments, the method comprises a gene therapy method e.g., administration of an isolated nucleic acid to inactivate a polypeptide. Engineering of such isolated nucleic acids by recombinant DNA or RNA methods is well within the ability of one skilled in the art. Administration of an isolated nucleic acid inactivating a polypeptide is encompassed by the expression “administering a therapeutically effective amount of a polypeptide inhibitor”. Gene therapy methods are well known in the art. See, e.g., WO96/07321 which discloses the use of gene therapy methods to generate intracellular antibodies. Gene therapy methods have also been successfully demonstrated in human patients. See, e.g., Baumgartner et al., Circulation 97: 12, 1114-1123 (1998), and more recently, Fatham, C. G. ‘A gene therapy approach to treatment of autoimmune diseases’, Immun Res. 18:15-26 (2007); and U.S. Pat. No. 7,378089, both incorporated herein by reference. See also Bainbridge J W B et al. “Effect of gene therapy on visual function in Leber's congenital Amaurosis”. N Engl J Med 358:2231-2239, 2008; and Maguire A M et al. “Safety and efficacy of gene transfer for Leber's Congenital Amaurosis”. N Engl J Med 358:2240-8, 2008.

There are two major approaches for introducing a nucleic acid inactivating a polypeptide (optionally contained in a vector) into a patient's cells; in vivo and ex vivo. For in vivo delivery the nucleic acid is injected directly into the patient, usually at the site where the polypeptide is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. Commonly used vectors for ex vivo delivery of the gene are retroviral and lentiviral vectors.

Preferred in vivo nucleic acid transfer techniques include transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. For review of the currently known gene marking and gene therapy protocols see Anderson et al., Science 256:808-813 (1992). See also WO 93/25673 and the references cited therein.

“Gene therapy” includes both conventional gene therapy where a lasting effect is achieved by a single treatment, and the administration of gene therapeutic agents, which involves the one time or repeated administration of a therapeutically effective DNA or mRNA. Oligonucleotides can be modified to enhance their uptake, e.g. by substituting their negatively charged phosphodiester groups by uncharged groups. Peptides of the present invention can be delivered using gene therapy methods, for example locally in a region of the brain, or systemically (e.g., via vectors that selectively target specific tissue types, for example, tissue-specific adeno-associated viral vectors). In some embodiments, primary cells (such as lymphocytes or stem cells) from the individual can be transfected ex vivo with a gene encoding any of the peptides of the present invention, and then returning the transfected cells to the individual's body.

One aspect of the invention provides a method of treating or preventing cardiovascular disease using an inhibitor of the invention. In one embodiment, the inhibitor of the invention can be used to suppress the onset of cardiovascular disease by inhibiting calpain in vascular endothelial cells.

The following are non-limiting examples of cardiovascular diseases that can be treated by the disclosed methods and compositions: arterial cardiovascular thromboembolic disorders, venous cardiovascular thromboembolic disorders, and thromboembolic disorders in the chambers of the heart; ahtherosclerosis; restensosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); first or recurrent myocardial infarction; acute myocardial infarction (AMI); myocardial infarction; non-Q wave myocardial infarction; non-STE myocardial infarction; coronary artery disease; cardiac ischemia; ischemia; ischemic sudden death; transient ischemic attack; stroke; atherosclerosis; peripheral occlusive arterial disease; venous thrombosis; deep vein thrombosis; thrombophlebitis; arterial embolism; coronary arterial thrombosis; cerebral arterial thrombosis; cerebral embolism; kidney embolism; pulmonary embolism; thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis; thrombosis resulting from atherosclerosis, surgery or surgical complications, prolonged immobilization, arterial fibrillation, congenital thrombophilia, cancer, diabetes, effects of medications or hormones, and complications of pregnancy; cardiac arrhytmias including supraventricular arrhythmias, atrial arrhythmias, atrial flutter, atrial fibrillation; other diseases listed in Heart Disease: A Textbook of Cardiovascular Medicine, 2 Volume Set, 6th Edition, 2001, Eugene Braunwald, Douglas P. Zipes, Peter Libby, Douglas D. Zipes.

The inhibitors of the invention can be administered alone or in combination with antioxidant agents. Examples of such agents include, but are not limited to ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite, pycnogenol, superoxide dismutase, pine bark grape seed complex, garlic, carotenoids, choline, metabisulfite, catechin, glangin, rutin, luteolin, morin, fisetin, silymerin, ascorbyl palmitate, apigenin, gingkolides, hesperitin, cyanidin, citrin, sodium bisulfite or mixtures thereof.

The inhibitors of the invention can be administered alone or in combination with protein kinase inhibitors, for instance, protein kinase C inhibitors. Examples of such inhibitors include any of a variety of bisindolylmaleimide compounds or indazolyl-substituted pyrroline compounds, such as those compounds disclosed in the following references, all of which are incorporated by reference herein in their entirety: Davis et al., FEBS Lett. 259(1):61-63 (1989); Twomey et al., Biochem. Biophys. Res. Commun. 171(3): 1087-1092 (1990); Toullec, et al., J. Biol. Chem. 266(24): 15771-15781 (1991); Davis et al., J. Med. Chem. 35:994-1001 (1992); Bit et al., J. Med. Chem. 36:21-29 (1993); U.S. Pat. Nos. 5,057,614, 5,936,084, and 6,284,783; International Publication Nos. WO 98/04551, WO 99/44606, WO 99/44607, WO 99/47518, and WO 02/46183; EP 0 940 141 A2.

The inhibitors of the invention can be administered alone or in combination with other cardiovascular disease therapeutic agents. Examples of such agents include, but are not limited to an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, a cyclooxygenase-2 inhibitor, an angiotensin system inhibitor, and/or combinations thereof.

### Pharmaceutical Compositions

The present invention includes pharmaceutical compositions comprising one or more inhibitors of calpain. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intratumoral, epidural, intracerebral, intracerebroventricular, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Hyperhomocysteinemia and Hyperglycemia Induce and Potentiate Endothelial Dysfunction Via μ-Calpain Activation

The results presented herein are based on the investigation of the joint effect of hyperhomocysteinemia (HHcy) and hyperglycemia (HG) on endothelial dysfunction (ED) and the underlying mechanisms.

Mild (22 μmol/L) and moderate HHcy (88 μmol/L) were induced in cystathionine β-synthase wild type (Cbs^{|/|}) and heterozygous deficient (Cbs^{-/|}) mice by a high methionine diet (HM). Hyperglycemia (HG) was induced by consecutive injection of streptozotocin. It was found that HG worsens HHcy and elevated Hcy levels to 55 μmol/L and 173 μmol/L in Cbs^{+/+} and Cbs^{-/+} mice fed a HM diet. Both mild and moderate HHcy aggravated HG-impaired endothelium-dependent vascular relaxation to acetylcholine, termed endothelial dysfunction (ED), which was completely abolished by endothelial nitric oxide synthase (eNOS) inhibitor L-NAME. HHcy potentiated HG-induced calpain activation in aortic endothelial cells isolated from CBS mice. Calpain inhibitors rescued HHcy/HG-induced ED via i.p. injection to mice or with ex vivo treatment of a mouse aorta. Moderate HHcy and HG induced μ-calpain activation in the mouse aorta. HHcy aggravated HG-induced aortic μ-calpain activation. Further, μ-calpain siRNA prevented HHcy/HG-induced ED and calpain activation in the mouse aorta. In addition, HHcy accelerated HG-induced superoxide production determined by DHE and 3-NT staining and urinary 8-isoprostane/creatinine measurements. Antioxidants rescued HHcy/HG-induced ED in mouse aortas and calpain activation in cultured human aortic endothelial cells (HAECs). Finally, HHcy potentiated HG-suppressed NO production and eNOS activity in HAECs which was prevented by calpain inhibitors or μ-calpain siRNA. HHcy aggravated HG-increased phosphorylation of eNOS at threonine 497/495 in the mouse aorta and HAECs. HHcy/HG induced eNOSp-Thr497/495 was reversed by μ-calpain siRNA and adenoviral transduced dominant negative PKCβ₂ in HAECs.

HHcy and HG induce ED and potentiate each other's effect via μ-calpain/PKCβ2 activation, resulting in eNOSp-Thr497/495 and eNOS inactivation.

The materials and methods used in these experiments are now described.

Experimental Animals and Sample Preparations

HHcy was induced in male Cbs^{-/+} or Cb^{+/+} mice (Jackson Laboratory) by feeding 8-week old mice with a newly designed high methionine (HM, 2%, 07794, Harlan Teklad) diet in which folic acid and B vitamins are reduced to the sufficient basal levels for 8 weeks (Zhang et al., 2009, Circulation 120:1893-1902). HG was induced by injection of streptozotocin (STZ) (i.p., 40 mg/kg BW/day), for 5 consecutive days at the age of 8-weeks. All animals received humane care in compliance with institutional guidelines and the “Guide for the Care and Use of Laboratory Animals” prepared by the Institute of Laboratory Animal Resources, Commission on Life Sciences, National Research Council.

After one week STZ injection, mice with blood glucose levels above 16.7 mmol/L were used. Blood samples were collected by cardiac puncture under anesthesia. 5 mm thoracic aorta were fixed in 4% buffered paraformaldehyde and cut with the microtome (HM310). The rest of aorta was used for Western blot. All samples were stored at −80° C. until assayed.

Endothelial function was assessed in the mouse thoracic aorta using a multiple-wire myograph (DMT 610) (Cheng et al., 2011, Blood 118:1998-2006; Cheng et al., 2007, Can J Physiol Pharmacol 85:404-412; Cheng et al., 2001, Hypertension 37:433-439). Role of NO, calpain and μ-calpain activation, and oxidative stress in HHcy- and/or HG-induced ED was examined in the presence of eNOS inhibitor, calpain inhibitors, calpain siRNA (calsiRNA) and antioxidants in vivo or ex vivo.

Isolation of Mouse Aortic Eendothelial Cells

Mouse aortic endothelial cells (MAECs) were isolated from Cbs^{-/+} mice using collagenase as previously described (Kobayashi et al., 2005, J Atheroscler Thromb 12:138-142), and identified with immunostaining of vWF and CD31.

Cell Culture

Human aortic endothelial cells (HAECs) were cultured in cell culture medium (M199, HyClone) as previously described (Wang et al., 1997, J Biol Chem 272:25380-25385). The HAECs were treated with DL-Hcy (200 or 500 μmol/L) and/or D-glucose (D-Glu, 25 mmol/L) for 24 or 48 hrs in the presence or absence of selected pharmacological inhibitors or siRNA or adenovirus.

Calpain Activity

“t-BOC” assay was used for measuring calpain activity in an intact single cell by treating MAECs or HAECs with t-butoxycarbonyl-leu-met-chloromethyl aminocoumarin (Boc-LM-CMAC, Molecular Probe) as previously described (Rosser et al., 1993, J Biol Chem 268:23593-23600).

Activated μ- and m-calpains autolytically cleave their NH₂-terminal ends (first 27 and 19 amino acids, respectively) (Li et al., 2004, Am J Physiol Regul Integr Comp Physiol 287:R1014-1030), resulting in the loss of NH₂ terminus domain antibody recognition, which was used as the indication of calpain activation (Stalker et al., 2005, Diabetes 54:1132-1140; Li et al., 2004, Am J Physiol Regul Integr Comp Physiol 287:R1014-1030; Scalia et al., 2011, Circulation research 108:1102-1111). Therefore, μ- and m-calpain activity were examined by Western blot analysis using polyclonal antibodies against NH₂ terminus domain of μ- and m-calpain (1:1000, ab28257, ab39167, Abcam) respectively. Total μ- and m-calpain expression was detected using polyclonal antibodies against domain IV (1:1000, ab39170, ab28259, Abcam) as previously described (Stalker et al., 2005, Diabetes 54:1132-1140; Li et al., 2004, Am J Physiol Regul Integr Comp Physiol 287:R1014-1030; Scalia et al., 2011, Circulation research 108:1102-1111).

RNA Interference in ECs and Aorta

HAECs were transfected with μ-calpain siRNA or control siRNA in accordance with the manufacturer's instructions (Santa Cruz). The transfection efficacy was determined by Western blot using rabbit polyclonal antibody against μ-calpain (sc7531, 1:1000, Santa Cruz).

In Situ Superoxide Production by DHE Staining

30 μm cross-section of fresh aorta segments were equilibrated for 30 minutes at 37° C. in Krebs buffer, and then incubated with dihydroethidium (DHE, 3 μmol/L) for 30 minutes at 37° C. in dark.

Peroxynitrite Production

3-nitrotyrosin (3-NT), a represented marker for peroxynitrite production, in the aorta was detected using an anti-3-NT monoclonal antibody (1:200, sc32757, Santa Cruz) and costained with 3,3′-diaminobenzidine (DAB) in 5 μm aortic sections as described previously (Cheng et al., 2011, Blood 118:1998-2006).

NO Production and eNOS Activity

HAECs treated with or without DL-Hcy and/or D-Glu for 48 hrs were stained with 4-Amino-5-Methylamino-2′,7′-Difluorofluorescein (DAF-FM). To explore the role of calpain, especially μ-calpain, the DL-Hcy/D-Glu-treated HAECs were also treated with MDL28170 (MDL, 20 μmol/L) and μ-calpain siRNA, respectively. HAECs treated with L-NAME (100 μmol/L) for 1 hr before DAF-FM staining were used as negative controls. eNOS activity in HAECs was determined by measuring the conversion of ³H-Arg to ³H-citrulline in intact cells as previous described (Jiang et al., 2005, Arterioscl Throm Vas 25:2515-2521).

Biochemical Determinations

Total plasma Hcy (t-Hcy) level was measured by mass spectrometer (Baylor Research Institute). Urinary 8-isoprostane level was determined by ELISA kit (Cayman) Blood glucose levels were determined in the blood collected from tail by a glucometer (HemoCue AB).

Statistical Analysis

Results are expressed as the mean±SEM. For statistical comparison of single parameters, unpaired-t test was used for two groups from same or each mouse strain. One-way ANOVA with Tukey adjustment was performed for multiple groups in each mouse strain. p<0.05 was considered to be significant.

The results of the experiments are now described.

### Plasma Total Hcy Levels, Blood Glucose and Body Weight

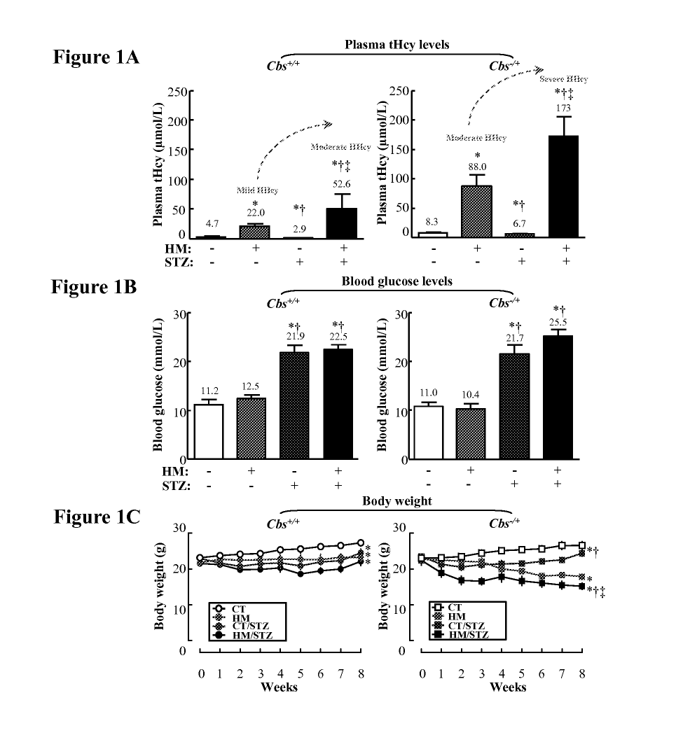

A HM diet was designed, which has folic acid and vitamin B levels in the sufficient basal levels (Zhang et al., 2009, Circulation 120:1893-1902), and induced mild HHcy in Cbs^{+/+} mice and moderate HHcy in Cbs^{-/+} mice (plasma tHcy: 22.0±3.3 and 88.0±19.5 μmol/L, respectively) (FIG. 1A). The HM diet induced moderate HHcy and severe HHcy in STZ-treated Cbs^{+/+} and STZ-treated Cbs^{-/+} mice, respectively (plasma tHcy: 52.7±26.5 and 173.0±32 μmol/L, respectively, p<0.05). HG was induced in the Cbs^{+/+} and Cbs^{-/+} mice by STZ injection (FIG. 1B). Body weight was significantly reduced in mice with either HHcy or HG alone (FIG. 1C), and to a greater extent in mice with HHcy/HG (p<0.05).

### HHcv Aggravated HG-Induced ED Via Suppressing NO Bioavailability

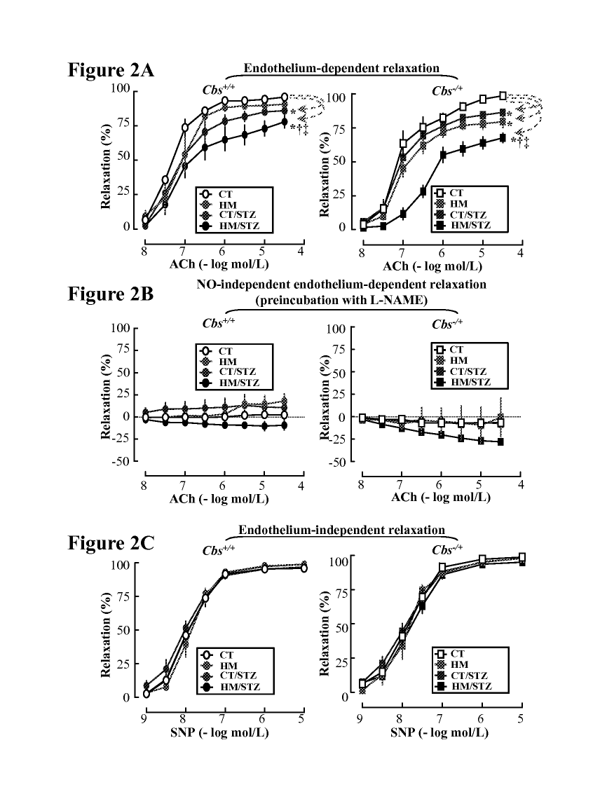

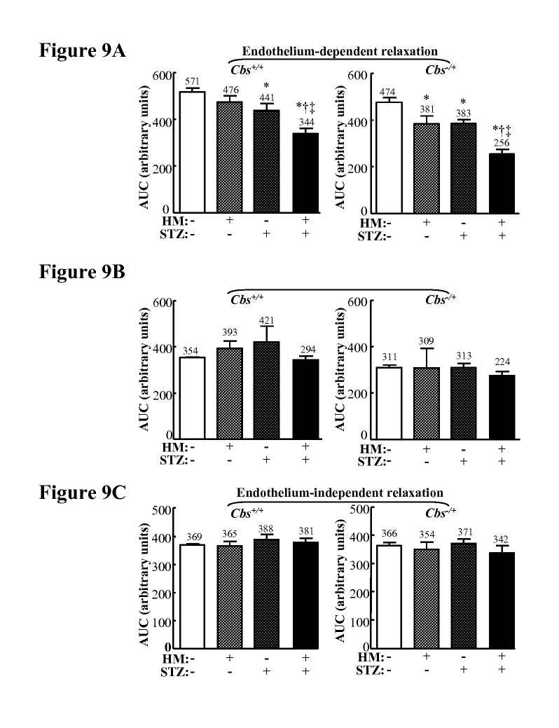

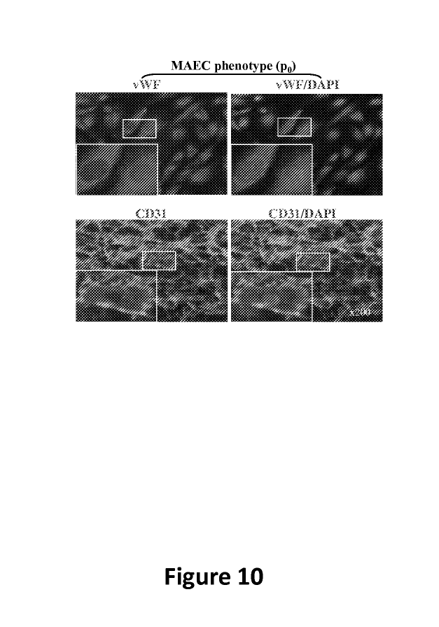

Mild HHcy in Cbs^{+/+} mice had no effect on endothelium-dependent vascular relaxation response to ACh (FIG. 2A., Left Panel), whereas moderate HHcy in Cbs^{-/+} mice impaired vascular relaxation to ACh (p<0.05, Right Panels in FIG. 2A and FIG. 9A). HG impaired vascular relaxation to ACh in both Cbs^{+/+} and Cbs^{-/+} mice (p<0.05). Notably, moderate HHcy in HHcy/HG-Cbs^{+/+} mice (Left Panel) potentiated HG-induced ED at a greater levels compared to either moderate HHcy lone-induced ED in Cbs^{-/+} mice (Right Panel) or HG alone-induced ED in HG-Cbs^{+/+} (Left Panel) or HG-Cbs^{-/+} mice (Right Panel). Severe HHcy further aggravated HG-induced ED in HHcy/HG-Cbs^{-/+} mice (p<0.05, Right Panels in FIG. 2A and FIG. 9A). The vascular relaxation response to ACh was completely abolished by eNOS inhibitor L-NAME in all groups (FIG. 2B and FIG. 9B).

Endothelium-independent vascular relaxation to SNP was similar in the aortas from all of groups (FIG. 2C and FIG. 9C).

### Calpain Activation Mediated HHcy/HG-Induced ED

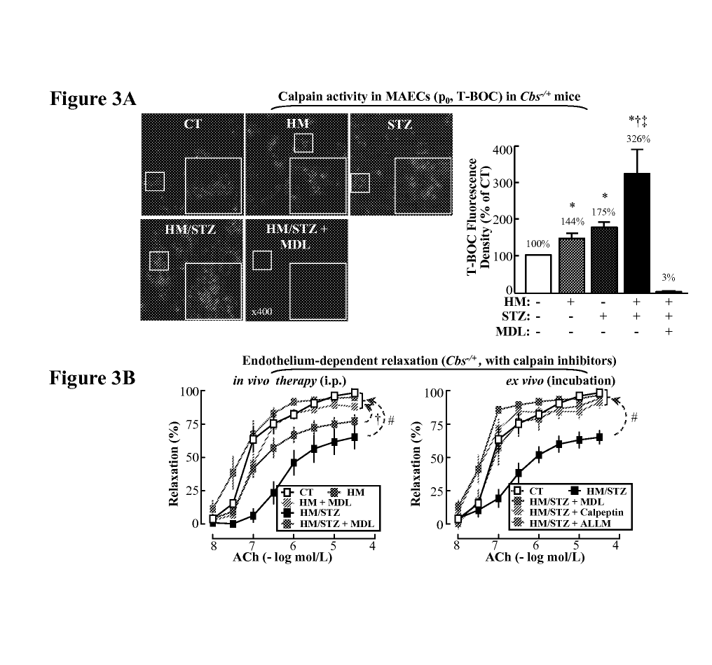

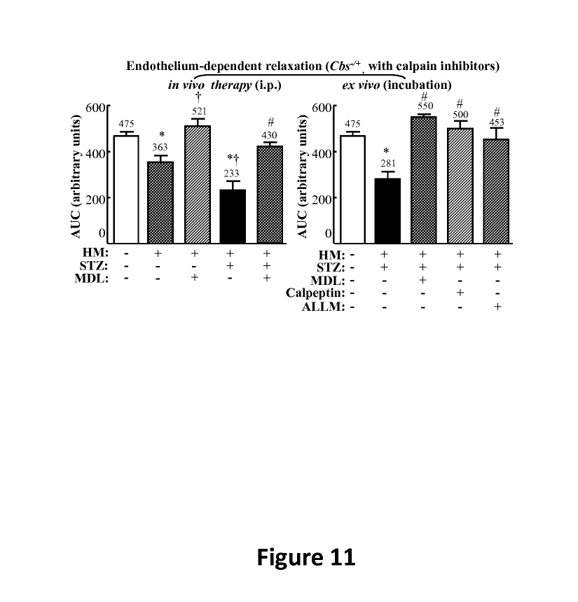

To examine the role of calpain in HHcy- and HHcy/HG-induced ED, calpain activity in MAECs isolated from Cbs^{-/+} mice was first examined by an enzymatic reaction using a calpain substrate, T-BOC. The MAECs were isolated by using collagenase. 90% of MAECs are vWF and CD31positive (FIG. 9). It was found that calpain activity was increased by 1.44- and 1.75-fold in MAECs isolated from Cbs^{-/|} mice with moderate HHcy or HG, respectively (p<0.05, FIG. 3A). However, in Cbs^{-/+} mice with combined severe HHcy and HG, calpain activity was increased by 3.26-fold (p<0.05). It was next tested whether calpain inhibitors can rescue HHcy- and HHcy/HG-induced ED. Calpain inhibitor MDL-28170 (MDL) was administrated daily to HHcy and HHcy/HG Cbs^{-/+} mice (2 mg/kg BW, i.p.) for 2 weeks and largely improved endothelial function in these mice (p<0.05, Left panels in FIG. 3B and FIG. 11). Moreover, preincubating the aortic rings with calpain inhibitors MDL, calpeptin, and ALLM (20 μmol/L) for 1 hour markedly improved aortic endothelium-dependent vascular relaxation to ACh in HHcy/HG-Cbs^{-/+} mice (p<0.05, Right panels in FIG. 3B and FIG. 11), demonstrating that calpain activation mediates HHcy and HHcy/HG-induced ED in mouse aortas.

### μ-Calpain Activation Mediated HHcy/HG-Induced ED

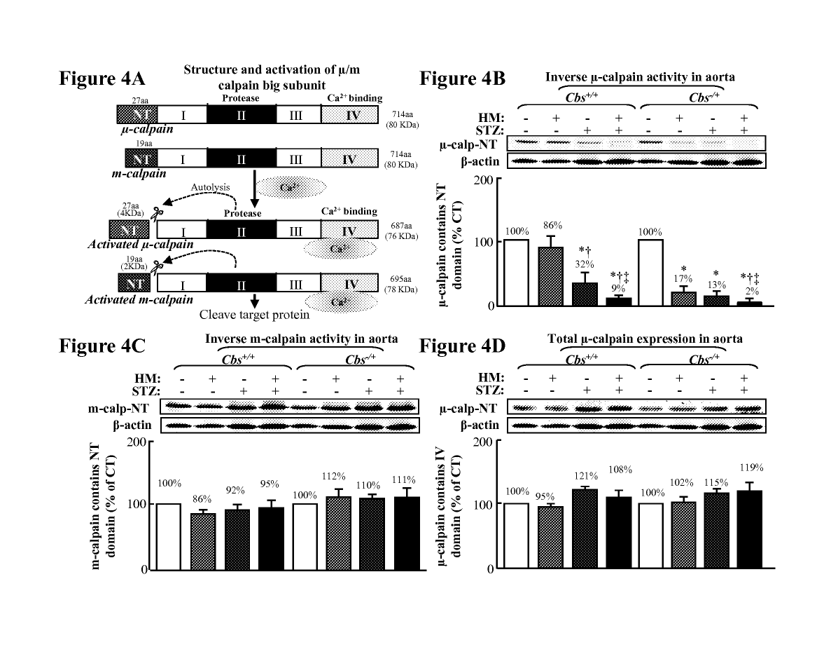

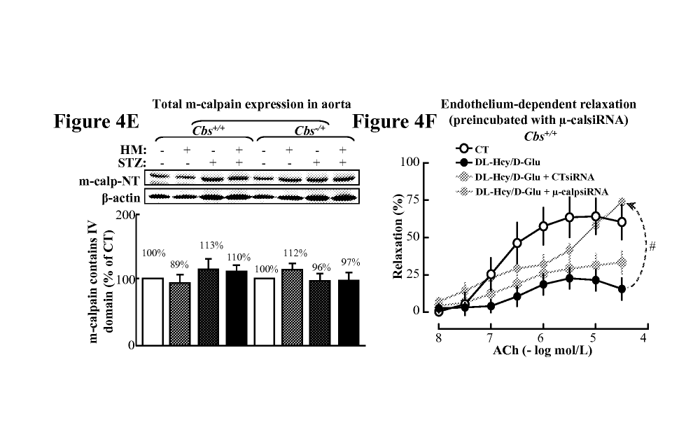

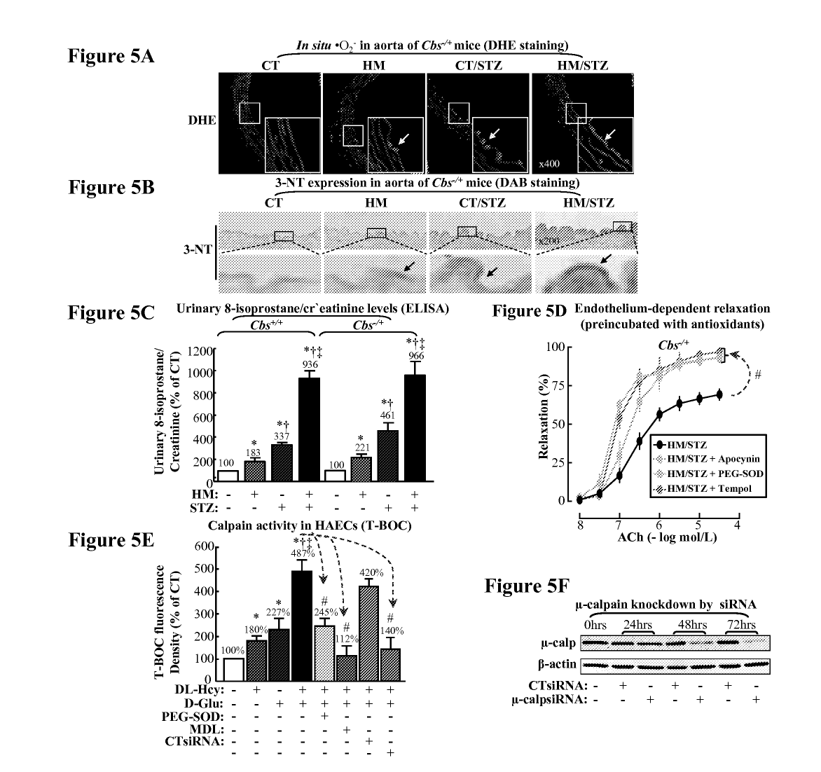

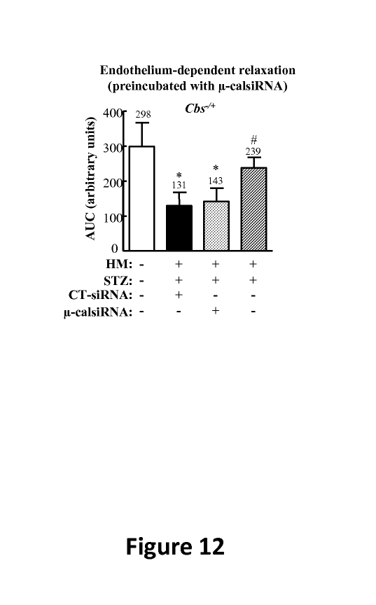

The 2 calpain isoforms, μ and m-calpain, are constitutively expressed in ECs (Li et al., 2004, Biochimica et biophysica acta 1691:91-103). When calpains are activated, they undergo autoproteolysis which removes N-terminal (NT) 27 (μ-calpain) and 19 (m-calpain) amino acids from the large subunit (catalytic subunit, FIG. 4A) (Li et al., 2004, Biochimica et biophysica acta 1691:91-103). This lowers levels of calpain large subunit NT and indicates higher degrees of proteolytic activity of calpain (inverse activity) (Goll et al., 2003, Physiol Rev 83:731-801). The effect of HHcy and/or HG on the activity of μ-calpain and m-calpain was determined by examining their NT large subunit in the mouse aorta as previously described (Goll et al., 2003, Physiol Rev 83:731-801). It was found that moderate HHcy in Cbs^{-/+} mice, but not mild HHcy in Cbs^{+/+} mice, significantly increased μ-calpain activity, as μ-calpain NT levels were reduced to 17% (p<0.05, FIG. 4B). HG also increased μ-calpain activity as μ-calpain NT levels were reduced to 32% in HG-Cbs^{+/+} mice, and 13% in HG-Cbs^{-/+} mice, respectively (p<0.05). HHcy/HG activated μ-calpain in the aortas from both HHcy/HG-Cbs^{+/+} and -Cbs^{-/+} mice to a greater extent (μ-calpain NT level was further reduced to 9% and 2%, respectively, p<0.05, FIG. 4B). HHcy and/or HG had no effect on m-calpain activity (FIG. 4C). Total μ- and m-calpain protein levels was further examined using an antibody against the stable domain IV of the large subunit, which recognizes all forms of μ- and m-calpain (with and without NT) (Goll et al., 2003, Physiol Rev 83:731-801). It was found that HHcy and/or HG had no effect on aortic total μ- and m-calpain protein levels (FIGS. 4D and 4E). Moreover, it was found that silencing the μ-calpain gene by μ-calpsiRNA significantly rescued HHcy/HG-induced ED in vitro (p<0.05, FIG. 4F and FIG. 12). Additionally, μ-calpsiRNA reversed HHcy/HG-induced calpain activation in HAECs (p<0.05, FIG. 5E).

### HHcy Aggravated HG-Induced Oxidative Stress

One of potential mechanisms leading to calpain activation is oxidative stress (Li et al., 2009, Free Radical Bio Med 46:51-61). O₂_{^{−}} production in the aorta CBS^{-/+} mice was examined with or without STZ injection and fed CT or HM diet by DHE staining of aortic cross-sections and it was found that moderate HHcy or HG alone markedly increased O₂_{^{−}} generation in the endothelium (FIG. 5A). Moderate HHcy potentiated HG-induced endothelial O₂_{^{−}} production to a greater extent. Because peroxynitrite (ONOO⁻) is a strong and relatively stable oxidant species capable of causing nitrosylation of tyrosine residues on key cellular proteins, the content of 3-NT in the aorta was also measured. It was found that moderate HHcy or HG increased 3-NT levels in the endothelium, which was dramatically elevated in HHcy/HG-Cbs^{-/|} mice (FIG. 5B). Moreover, HHcy increased urinary 8-isoprostane, a prostaglandin-like compound formed from the free radical-catalyzed peroxidation of arachidonic acid, by 1.8- and 2.2-fold in HHcy-Cbs^{+/+} and HHcy-Cbs^{-/+} mice, respectively (p<0.05, FIG. 5C). HG increased urinary 8-isoprostane by 3.4 and 4.6-fold in HG-Cbs^{+/+} and HG-Cbs^{-/+} mice, respectively (p<0.05, FIG. 5C). HHcy dramatically increased HG-induced urinary 8-isoprostane by 9.4- and 9.7-fold in HHcy/HG-Cbs^{+/+} and HHcy/HG-Cbs^{-/+} mice levels, respectively.

### Oxidative Stress Mediated HHcy/HG-Induced ED

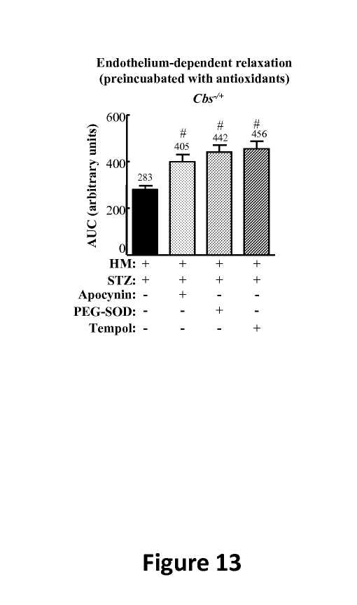

To investigate the role of oxidative stress in HHcy/HG-induced ED, the relaxation response to ACh was examined in aortic rings from HHcy/HG-Cbs^{-/+} mice preincubated with antixoidants for 1 hr. It was found that endothelium-dependent vascular relaxation to ACh was significantly improved by PEG-SOD, Tempol, and apocynin (p<0.05, FIG. 5D and FIG. 13).

### HHcy Aggravated HG-Induced μ-Calpain Activation Via Oxidative Stress in HAECs

To address the human relevance, effects of HHcy and/or HG on calpain was examined, especially μ-calpain activity in HAECs. Calpain activity was increased by 1.8- and 2.3-fold in HAECs treated with DL-Hcy (500 μmol/L) and D-Glu (25 mmol/L) for 48 hrs, respectively (FIG. 5F, p<0.05). The combination of DL-Hcy and D-Glu increased calpain activity by 4.9-fold (FIG. 5E, p<0.05). Antioxidant PEG-SOD significantly decreased HHcy/HG-induced calpain activation by 49% (p<0.05). To study the role of μ-calpain in HHcy/HG-induced calpain activation, μ-calpain gene in HAECs was knocked down by μ-calpsiRNA which completely deleted the μ-calpain gene in HAECs after 72 hr transfection (FIG. 5E). μ-calpsiRNA rescued HHcy/HG-induced calpain activation (FIG. 5F, p<0.05).

HHcy Aggravated HG-Induced NO Reduction and eNOS Inactivation Via Activation of μ-calpain

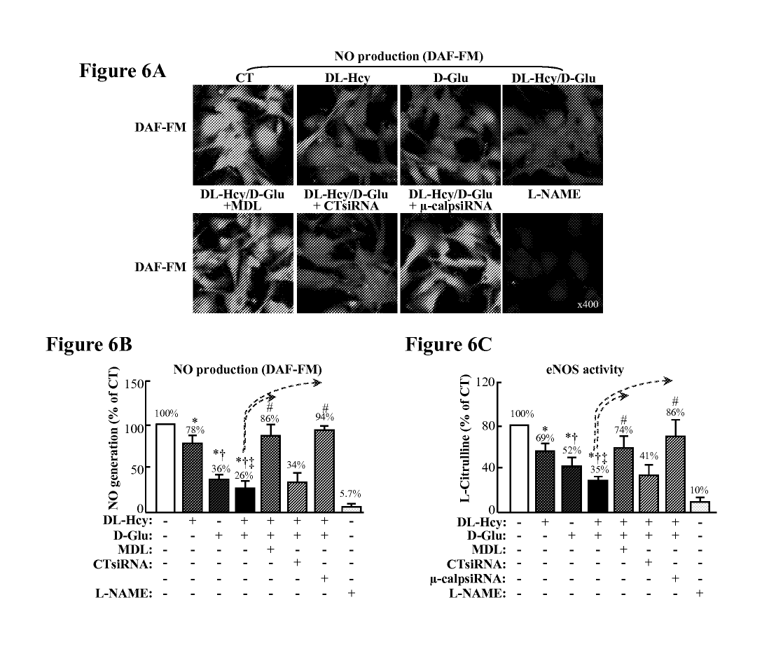

To assess the effect of HHcy and HG on NO production and eNOS activity, NO production and eNOS activity was examined by DAF staining and citrulline conversion in HAECs. It was found that DL-Hcy (500 μmol/L) and D-Glu (25 mmol/L) for 48 hrs significantly reduced NO generation to 78% and 36% of the control (p<0.05, FIGS. 6A and 6B) and eNOS activity to 69% and 52% (p<0.05, FIG. 6C) in HAECs, respectively. The combination of DL-Hcy plus D-Glu further reduced NO production and eNOS activity to 26% and 35%, respectively (p<0.05). HAECs were further treated with calpain inhibitor MDL to test the role of calpain activation on HHcy- and HG-induced NO reduction and eNOS inactivation. It was found that NO production and eNOS activity was largely improved by MDL from 26% and 35% to 86% and 74% in HHcy/HG-treated HAECs, respectively (p<0.05, FIG. 6). Further, μ-calpsiRNA markedly improved NO production from 26% to 94% and eNOS activity from 26% to 86% in HAECs treated with DL-Hcy/D-Glu (p<0.05, FIG. 6). NOS inhibitor L-NAME diminished NO generation and eNOS activity to 5.7% and 10%, respectively.

HHcy Aggravated HG-Induced eNOS-pThr495

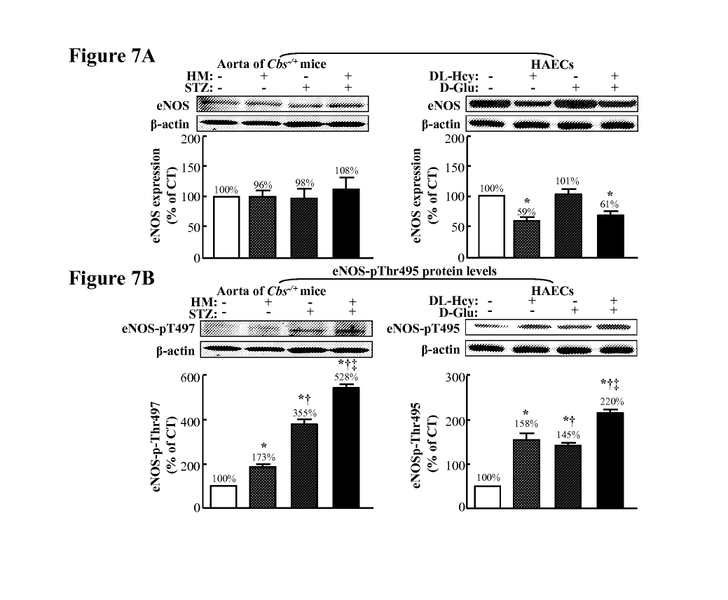

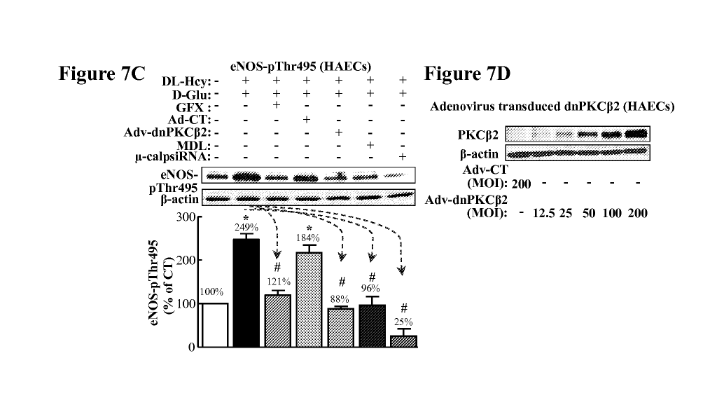

The effect of HHcy/HG on eNOS expression was next examined and it was found that neither HHcy nor HG influenced eNOS expression in the mouse aorta (p<0.05, FIG. 7A, Left Panel). In good accordance with a previous study (Jiang et al., 2005, Arterioscl Throm Vas 25:2515-2521), it was found that DL-Hcy (500 μmol/L, 48 hrs) decreased eNOS protein levels by 59% (FIG. 7B, Right Panel), whereas, D-Glu had no effect in HAECs both in the presence or absence of DL-Hcy.

Phosphorylation of eNOS at threonine 497 (rodent) or 495 (human) (eNOS-pThr497/495) is a negative regulatory site and is phosphorylated by PKC—α substrate of calpain (Inoue et al., 1977, J Biol Chem 252:7610-7616; Takai et al., 1977, Biochem Bioph Res Co 77:542-550). The effect of HHcy/HG on eNOS-pThr495/497 in mouse aortas and HAECs was examined. The role of PKC and calpain in HHcy/HG regulated eNOS-pThr495 in HAECs was also tested by using pharmacological inhibitors and gene silencing approaches. It was previously found that Hcy increased eNOS-pThr495 in HAECs (Jiang et al., 2005, Arterioscl Throm Vas 25:2515-2521). Here, supporting data is provided showing that HHcy and HG increased eNOS-pThr497/495 levels by 1.7- and 3.6-fold in the aorta of HHcy-Cbs^{-/+} and HG-Cbs^{-/+} mice (p<0.05, FIG. 7B Left Panel), and 1.6- and 1.5-fold in HAECs treated with DL-Hcy (500 μmol/L) and D-Glu (25 mmol/L) (p<0.05, FIG. 7B Right Panel), respectively. The combination of HHcy and HG markedly increased eNOS-pThr497/495 by 5.4-fold in the aorta of HHcy/HG Cbs^{-/+} mice and 2.2-fold in the HAECs. Importantly, HHcy/HG-induced eNOS-pThr495 was completely rescued by non-selective PKC inhibitor GFX and calpain inhibitor MDL (p<0.05, FIG. 7C) indicating that activation of PKC and calpain regulates HHcy/HG-induced eNOS-pThr497/495. PKCβ₂ has been shown to be induced in diabetic vascular tissues. To examine the role of PKCβ₂ in the regulation of eNOS-pThr497/495 in HAECs under HHcy/HG condition, PKCβ₂ gene in HAECs was first silenced by transfecting adenoviral transduced dominant negative PKCβ₂ (Adv-dnPKCβ₂, FIG. 7D). It was found that HHcy/HG-induced eNOS-pThr495 was significantly reversed by Adv-dnPKCβ₂ (p<0.05, FIG. 7C). Moreover, μ-calpsiRNA also markedly HHcy/HG-induced eNOS-pThr495 (p<0.05, FIG. 7C).

### HHcv and HG Induce and Potentiate ED Via μ-Calpain Activation

The individual and combined effects of HHcy and HG on vascular function in the thoracic aorta of Cbs^{+/+} and Cbs^{-/+} mice fed a HM diet and injected with STZ was investigated. Three major novel findings are reported. Firstly, HG worsens HHcy. Secondly, HHcy and HG induce ED and potentiate each other's effect via μ-calpain and PKCβ2 activation, resulting in eNOSp-Thr497/495 and eNOS inactivation. It is proposed that μ-calpain activation causes ED and contributes to heightened cardiovascular risk seen in both Type 1 and Type 2 diabetes patients with HHcy.

HHcy has been considered as an independent risk factor of CVD since 1969. Accumulative evidence indicate that patients with diabetes have a high prevalence of HHcy, which is positively correlated with diabetes-induced cardiovascular mortality and morbidity (Hofmann et al., 1998, Diabetes care 21:841-848; Okumura et al., 2003, Metabolism: Clinical and Experimental 52:1517-1522). HHcy aggravated ED and arterial stiffness in patients with Type 2 diabetes mellitus (Chousos et al., 2010, Diab Vasc Dis Res 7:186-194; Doupis et al., 2010, Exp Clin Endocrinol Diabetes 118:453-458). Folic acid supplementation improved endothelium-dependent vascular relaxation responses to ACh in the aorta of db/db mice (Type 2 diabetes mouse model) via the NO pathway (Seto et al., 2010, J Nutr Biochem 21:872-880). Here, it is demonstrated that Hcy dose-dependently aggravated HG-induced ED via activation of μ-calpain, indicating that HHcy is an important risk factor for ED in diabetes.

NO is a crucial player in vascular homeostasis. NO is synthesized within ECs during conversion of L-arginine to L-citrulline by eNOS. Loss of NO bioavailability and eNOS activity has been implicated in the pathophysiology of several disease states including coronary artery disease, hypertension, heart failure, HHcy, and diabetes. In the present study, it was found that both HHcy and HG impaired the endothelium-dependent vascular relaxation response to ACh. Notably, Hcy dose-dependently aggravated HG-induced ED. Moreover, eNOS inhibitor L-NAME completely blocked the endothelium-dependent vascular relaxation response to ACh in the aorta from mice with HHcy and/or HG, demonstrating that decrease of NO bioavailability plays a major role in these impairments.

Calpains are a family of calcium-dependent cysteine-proteases. Activation of calpain has been implicated in the pathogenic changes in diabetes, such as platelet aggregation, neurovascular dysfunction, and cardiomyocyte apoptosis (Randriamboavonjy et al., 2008, Circulation 117:52-60; Nangle et al., 2006, Eur J Pharmacol 538:148-153). Recently, increased calpain activation was reported to be related to HG-induced microvascular inflammatory responses (Stalker et al., 2005, Diabetes 54:1132-1140; Stalker et al, 2003, Official Publication of the Federation of American Societies for Experimental Biology 17:1511-1513). Here it was demonstrated that HHcy aggravated HG-induced ED via activation of calpain in mouse aortas since calpain inhibitor MDL, ALLM, or calpeptin markedly improved vascular relaxation response to ACh in the aorta from HHcy and HHcy/HG mice.

In the calpain family, μ- and m-calpain are well-characterized players within 15 members which are expressed abundantly in ECs (Goll et al., 2003, Physiol Rev 83:731-801). The μ and m-calpain consist of two subunits: a distinct larger (about 80 kDa) subunit and a common smaller subunit (30 kDa). The larger subunits can be divided into four domains (FIG. 4). The second domain is a cysteine protease domain whereas the fourth one is a Ca²⁺-binding domain. The smaller subunits can be divided into domain V (N-terminal glycine-clustering hydrophobic domain) and VI (C-terminal glycine rich domain). Therefore, the proteinase activity of calpains is ascribed to the large subunit. Calpains are activated and begin to autolyse after binding with Ca²⁺, thus removing the N-terminal 27 (μ-calpain) or 19 (m-calpain) amino acids from the large subunit (FIG. 4) (Li et al., 2004, Am J Physiol Regul Integr Comp Physiol 287:R1014-1030), resulting in the loss of NH₂ terminus domain antibody recognition, which has been used as the indication of reverse calpain activity extensively (Stalker et al., 2005, Diabetes 54:1132-1140; Li et al., 2004, Am J Physiol Regul Integr Comp Physiol 287:R1014-1030; Scalia et al., 2011, Circulation research 108:1102-1111). Here it was demonstrated that HHcy/HG increased activity of μ, but not m-calpain, in mouse aortas, to a greater extent compared to that from mice with HHcy or HG alone. Moreover it was reported that μ-calpain activation plays a major role in HHcy/HG-induced ED since μ-calsiRNA significantly improved vascular response to ACh in the aortas treated with DL-Hcy/D-Glu for 48 hrs. Taken together, it was demonstrated that μ-calpain activation regulates HHcy/HG-induced ED in mouse aortas via decrease of NO bioavailability.

Calpains tightly regulate their respective substrates through limited proteolytic cleavage. Activation of calpain leads to disruption of eNOS (Dong et al., 2008, J Cell Mol Med 13:2899-2910; Youn et al., 2009, Circ Res 104:50-59), thus decreasing NO bioavailability. Here it was found that HHcy decreased eNOS expression by 59% in HAECs treated with DL-Hcy (500 μmol/L, 48 hrs), whereas D-Glu (25 mmol/L, 48 hrs) did not significantly affect eNOS expression. eNOS expression was not changed significantly in the aorta from HHcy-, HG- or HHcy/HG CBS^{-/+} mice. Notably, the combination of HHcy and HG did not further decrease eNOS expression both in HAECs and mouse aortas, suggesting that the degradation of eNOS by calpain is not involved in HHcy/HG-induced ED.

Thr497 (rodent)/495 (human) in the CaM-binding domain is one of key regulatory sites for eNOS activity. eNOS Thr497/495 can be phosphorylated by AMP-activated kinase and PKC, resulting in reduced eNOS catalytic activity. PKC is known to be an important signaling molecule in diabetic ED (Geraldes et al, 2012, Circ Res 106:1319-1331). It was previously reported that PKC activation mediates HHcy-induced eNOS-pThr495 and eNOS inactivation in HAECs, and proposed that PKC induced eNOS-pThr495/497 may play a major role in HHcy-induced ED in microvasculature (Jiang et al., 2005, Arterioscl Throm Vas 25:2515-2521). In the present study, it was found that HHcy aggravated HG-induced eNOS-pThr495 which is blocked by PKC inhibitor GF109203X (GFX). Within the PKC family, PKCβ has received much attention since it was first shown to be preferentially upregulated in diabetic vascular tissue (Inoguchi et al., 1992, P Natl Acad Sci USA 89:11059-11063). It was suggested that PKCβ₂ activation mediates HG-induced ED and cardiomyocyte apoptosis (Smolock et al., 2011, Arterioscl Throm Vas 31:289-296; Randriamboavonjy et al., 2008, Circulation 117:52-60). Here, for the first time, it was demonstrated that PKCβ₂ suppression by Adv-dnPKCβ₂ transduction rescued HHcy/HG-induced eNOS-pThr495 in HAECs.

PKC was originally discovered as a kinase cleaved and activated by calpains (Inoue et al., 1977, J Biol Chem 252:7610-7616) which tightly regulate their respective substrates through limited proteolytic cleavage. However, recent studies also suggested that calpain can serve as a downstream target of PKC signaling. PKC induces calpain phosphorylation in cultured cancer cells (Xu et al., 2006, J Biol Chem 281:4457-4466). PKC inhibitor BIM-1 decreased calpain activity in mouse microvascular endothelial cells (Smolock et al., 2011, Arterioscl Throm Vas 31:289-296). Nevertheless, here it was shown that both calpain inhibition by using MDL or μ-calsiRNA, and PKCβ₂ inhibition by Adv-dnPKCβ₂ rescued HHcy/HG-induced eNOS-pThr495. These data suggest that both μ-calpain and PKCβ₂ activation contribute to HHcy/HG-induced eNOS inactivation and NO reduction. The molecular details for interaction between PKCβ₂ and calpain activation under HHcy/HG condition are warranted.

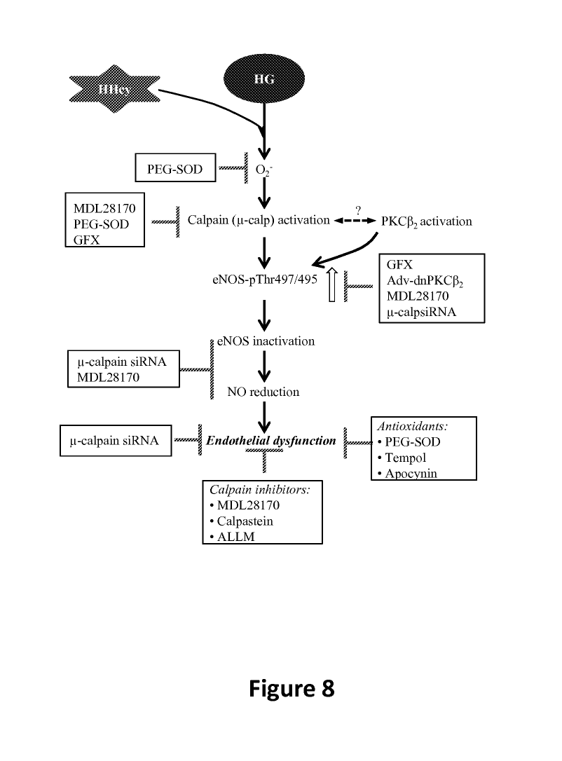

Oxidative stress has been suggested to be one of the major factors for calpain activation. ROS induce calpain activation in retinal photoreceptor cells, cardiomyocytes, and pulmonary microvascular ECs (Li et al., 2009, Free Radical Bio Med 46:51-61; Sanvicens et al., 2004, J Biol Chem 279:39268-39278; Hu et al., 2009, Microvasc Res 78:33-39). Inhibition of NADPH oxidase or ROS production significantly prevented calpain activation in pulmonary microvascular ECs (Hu et al., 2009, Microvasc Res 78:33-39). Oxidative stress-induced calpain activation may be related to cysteine oxidation of plasma membrane calcium ATPase (Zaidi et al., 2010, World J Biol Chem 1:271-280) and free radical-activated L-type voltage-sensitive calcium channels induced Ca²⁺ overloading (Kowara et al., 2006, Brain Res 1119:40-49). Moreover, ONOO⁻ is responsible for the cleavage of the N-terminus of latent u-calpain (Randriamboavonjy et al., 2008, Circulation 117:52-60). Recent studies demonstrated that Hcy induced the activation and mitochondrial translocation of μ-calpain, via increasing mitochondrial oxidative stress in rat heart microvascular ECs (Moshal et al., 2006, Am J Physiol Heart Circ Physiol 291:H2825-2835). However, oxidative stress induced μ-calpain activation has not been connected with any vascular dysfunction. It was found that HHcy aggravated HG-induced O₂_{^{−}} generation in the endothelium of the aorta and the whole body of mouse. Antioxidant PEG-SOD, Tempol, and apocynin improved HHcy/HG-induced ED. Moreover, it was shown that HHcy/HG-induced calpain activation was prevented by antioxidant PEG-SOD, demonstrating that oxidative stress is responsible for, at least partially, HHcy/HG-induced calpain activation (FIG. 8).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for treating endothelial dysfunction in a subject having hyperhomocysteinemia, hyperglycemia, or a combination of hyperhomocysteinemia and hyperglycemia, the method comprising administering to the subject a therapeutically effective amount of a composition comprising at least one inhibitor of calpain.

2. The method of claim 1, wherein the inhibitor is selected from the group consisting of a small interfering RNA (siRNA), a microRNA, an antisense nucleic acid, a ribozyme, an expression vector encoding a transdominant negative mutant, an antibody, a peptide, a chemical compound, and a small molecule.

3. The method of claim 2, wherein the peptide is at least one selected from the group comprising MDL28170, calpeptin, and ALLM.

4. The method of claim 2, wherein said siRNA is μ-calpsiRNA.

5. The method of claim 1, wherein said composition further comprises a pharmaceutically acceptable excipient.

6. The method of claim 1, wherein said composition is administered in combination with another therapeutic agent.

7. The method of claim 6, wherein said another therapeutic agent is an antioxidant.

8. The method of claim 7, wherein said antioxidant is at least one selected from the group comprising PEG-SOD, Tempol, and Apocynin.

9. The method of claim 6, wherein said another therapeutic agent is a PKC suppressor.

10. The method of claim 9, wherein said PKC suppressor is at least one selected from the group comprising GFX and dominant negative PKCβ2 adenovirus.

11. A method for detecting the onset of cardiovascular disease in a subject having hyperhomocysteinemia or a combination of hyperhomocysteinemia and hyperglycemia, said method comprising obtaining in a biological sample the quantity of calpain N-terminal catalytic subunits, obtaining in the same biological sample the quantity of total calpain protein levels, and transforming the quantities obtained into a therapeutically relevant ratio of calpain N-terminal catalytic subunits to total calpain protein levels.

12. The method of claim 11, wherein the quantity of catalytic subunits obtained is the quantity of N-terminal catalytic subunits of μ-calpain and the quantity of total calpain protein level obtained is the quantity of total μ-calpain protein level.

13. The method of claim 11, wherein the ratio is less than 40%.

14. The method of claim 11, wherein the ratio is less than 20%.

15. The method of claim 11, wherein the ratio is less than 10%.

16. The method of claim 11, wherein the ratio is less than 5%.

17. A kit for diagnosing or monitoring the onset of cardiovascular disease in a subject wherein the quantity of calpain N-terminal catalytic subunits is obtained from a biological sample from said subject, the quantity of total calpain protein levels is obtained from the same biological sample from said subject, and the quantities obtained are transformed into a therapeutically relevant ratio of calpain N-terminal catalytic subunits to total calpain protein levels.

18. The kit of claim 17, wherein the quantity of catalytic subunits obtained is the quantity of N-terminal catalytic subunits of μ-calpain and the quantity of total calpain protein level obtained is the quantity of total μ-calpain protein level.
